(19) United States
(12) Patent Application Publication
(10) Pub. No.: US 2023/0380685 A1
(43) Pub. Date: Nov. 30, 2023

(54) WEARABLE AND NON-CONTACT INTRAOCULAR PRESSURE (IOP) MEASUREMENT AND MONITORING SYSTEM

(71) Applicant: GlaucoT Teknoloji AS, Istanbul (TR)

(72) Inventors: Abdulkadir ODUNCU, Istanbul (TR); Veysel OZKAPICI, Istanbul (TR); Alperen ACEMOGLU, Istanbul (TR)

(73) Assignee: GlaucoT Teknoloji AS, Istanbul (TR)

(21) Appl. No.: 17/751,664

(22) Filed: May 24, 2022

(51) Int. Cl. A61B 3/16; A61B 5/00; G16H 50/30

(52) U.S. Cl. CPC A61B 3/16; A61B 5/6821; A61B 5/6803; G16H 50/30; A61B 5/742; A61B 3/107

(57) ABSTRACT

A system and method for non-invasively and continuously measuring an intraocular pressure, includes a wearable device designed to be worn by a user and forms an air chamber between the wearable device and the eyes of the user. The wearable device includes flexible materials to seal the air chamber around eye cavities of the user and further includes imaging sensors for collecting 2D and 3D data of the user's eyes; a pressure controller to modify an air pressure inside the air chamber with an air pump, wherein the pressure controller can be either embedded on the wearable device or is connected to the wearable device through a flexible pressure pipe; and a central controller for constructing a model of the user's eyes using 3D imaging techniques based on the collected 2D and 3D data and output intraocular pressure calculations based on the model.

### TECHNICAL FIELD

This instant invention relates to the field of a non-invasive measurement of an intraocular pressure with a wearable device.

### BACKGROUND

Glaucoma is the most frequent cause of irreversible blindness. Many people over 40 years old suffer from glaucoma disease. The risk factors for causing Glaucoma disease includes intraocular pressure, neurodegeneration and vascular insufficiency. Within these factors, intraocular pressure is one of the most important risk factors, and studies have found intraocular pressure to be controllable. Aqueous humor that creates the intraocular pressure is generated by the ciliary body. Unbalance between the generation of the aqueous humor and drainage to the out of the eye globe causes an increase in the intraocular pressure. Long term increase of the intraocular pressure causes damage on the optical nerve and, consequently, glaucomatous vision loss.

Approximately, 50% of the glaucoma patients are not aware of their disease. The most important reason is that there are no symptoms until the end stage of the disease. After the diagnosis of the glaucoma, patients start to use different drugs with many side effects. During the drug use of a glaucoma patient, it is really important to monitor the variation of the diurnal intraocular pressure. Monitoring the intraocular pressure enables controlling the intraocular pressure and lowering the pressure on the optical nerve, which are crucial during the progression of the disease.

Conventionally, there are several types of tonometry for measuring intraocular pressures, includes:
1 Applanation tonometry
   1.1 Goldmann and Perkins applanation tonometry
   1.2 Non-Contact Tonometry
      1.2.1 Air Puff Tonometer
      1.2.2 Ocular Response Analyzer
2 Indentation tonometry
   2.1 Schiotz Tonometer
   2.2 Pneumotonometer
   2.3 Tono-Pen
3 Rebound tonometry
4 Pascal Dynamic Contour Tonometer
5 Continuous IOP Monitoring

Today, intraocular pressure monitoring is possible at home or in hospital with methods that require contact with the eye globe. However, the devices that can be used at home have several problems such as direct contact with the eye, difficult to use, and requiring long training to get correct measurements.

Current systems for measuring IOP use either air puff or a palpation on the eye globe. These methods have disadvantages such as i) difficulty in home use, ii) infection risk, iii) possible cornea damage, iv) limited use for yielding diurnal IOP trends.

The IOP measurement techniques have not changed for almost a century. The traditional methods like the Goldmann applanation tonometer (GAT) is still the clinical reference for IOP measurements. However, the measurement procedure is uncomfortable and carries infection risks through direct eye contact. Due to negative effects of the required anesthetics and the mechanical load on the cornea, the number of daily measurements is limited to about 5. This method is therefore of limited use for yielding diurnal IOP trends which is very important for Glaucoma patients. It also requires a physician for the measurement, which limits the GAT.

These drawbacks also apply to the Schiotz Tonometer (e.g. Rudolf Riester GmbH, Jungingen, Germany), where the measurement uncertainty is larger than with GAT. The Pascal dynamic contour tonometer (Ziemer Ophthalmic Systems AG, Port, Switzerland), which uses a concave contact area that indents the cornea only a few microns and measures the remaining corneal tension with a pressure sensor, is less prone to systematic effects of the corneal thickness, but still requires anesthetics and a physician for the measurement. The Ocuton S tonometer (EPSa GmbH, Saalfeld, Germany) can be considered a self-tonometer, which is based on the same measurement principle as the GAT. The disadvantage of the Ocuton S is the high measurement uncertainty of up to 10 mm Hg that does not allow acquiring reliable diurnal IOP trends and the contact to the cornea which brings infection risks. There is currently no available contact tonometer for home use and self-measurements.

Related reference WO2017132165 discloses non-invasively monitoring, measuring, and estimating intraocular pressure. It teaches a diagnostic method capturing high resolution imaging of the sclera of one or both of a patient's eyes by digital photography or videography. The hardware employed may be for two-dimensional (2D) or three-dimensional (3D) imaging. In this disclosure, blood vessel patterns are used to observe the changes on the eye globe. It also introduces a deflection based intraocular pressure measurement. However, it requires a reference value that is pre-measured with standard methods. Furthermore, it does not use any kind of excitation, and it does not explain how sensors can get stable measurements when a hand-held device is used.

Another related reference U.S. Pat. No. 6,673,014 discloses noninvasive methods and apparatuses measuring the intraocular pressure (IOP) of the eye using vibratory excitation are disclosed. Prior art methods teach that the natural frequencies of the eye vary as a function of the IOP, with each natural frequency being zero at zero IOP. This disclosure recognizes that the eye has different and separate classes of natural frequencies that vary as a function of the IOP, which have non-zero values for a zero value of IOP, and which have curves that extrapolate to negative IOPs to obtain zero values of frequency. This disclosure further teaches measuring a first natural frequency of this class at an unknown IOP value, and thereafter compares it to one or more known values of the first natural frequency measured at corresponding known IOPs to estimate a value of the unknown IOP. This disclosure uses goggles to create an air chamber to apply pressure on the eye globe. However, this method assumes that the natural frequency of the eye changes with intraocular pressure.

Another related reference U.S. Pat. No. 7,909,765 discloses a method and apparatus for measuring intraocular pressure of an eye use an empirically derived function wherein an inward applanation pressure P1 and an outward applanation pressure P2 obtained during a corneal deformation cycle caused by a fluid pulse are separately weighted so as to minimize cornea-related influence on the intraocular pressure value calculated by the function. The function is optimized, at least in part, to minimize change in calculated IOP between measurements made before surgical alteration of the cornea and measurements made after surgical alteration of the cornea. However, an applanation method is used for measuring the intraocular pressure and a fluid pulse is used to create an indent in the cornea.

Related reference WO2016162653 discloses a device for non-invasive determination of intra-ocular pressure comprising a pressure chamber with a flexible membrane for engaging with the cornea of a subject's eye and a strain gauge, wherein the pressure chamber may be internally pressurized and thereby exerts a pressure via the membrane upon both the cornea and the strain gauge that result in a deflection of the cornea and strains being generated at the strain gauge, which, in turn, enables the ability to establish a correlation between inter ocular pressure and strains. However, it requires direct contact with the cornea, and a pressurized chamber in front of the cornea is created. This chamber has a strain gauge on it, and a relation between the strain values and intraocular pressure is established.

Related reference U.S. Pat. No. 7,403,805 discloses a contact device placed on the eye in order to detect physical and chemical parameters of the body as well as the non-invasive delivery of compounds according to these physical and chemical parameters, with signals being transmitted continuously as electromagnetic waves, radio waves, infrared and the like. One of the parameters to be detected includes non-invasive blood analysis utilizing chemical changes and chemical products that are found in the conjunctiva and in the tear film. A transensor mounted in the contact device laying on the cornea or the surface of the eye is capable of evaluating and measuring physical and chemical parameters in the eye including non-invasive blood analysis. The system utilizes eye lid motion and/or closure of the eye lid to activate a microminiature radio frequency sensitive transensor mounted in the contact device. The signal can be communicated by wires or radio telemetered to an externally placed receiver. The signal can then be processed, analyzed and stored. Several parameters can be detected including a complete non-invasive analysis of blood components, measurement of systemic and ocular blood flow, measurement of heart rate and respiratory rate, tracking operations, detection of ovulation, detection of radiation and drug effects, diagnosis of ocular and systemic disorders and the like. However, this disclosure requires contact with the eye for measuring the chemical changes, chemical products, blood components, and parameters with a mid-infrared detecting method.

Another related reference U.S. Pat. No. 10,386,640 discloses a health system to be used in various healthcare applications, e.g., for patient diagnostics, monitoring, or therapy. The health system may comprise a light generation module to transmit light or an image to a user, one or more sensors to detect a physiological parameter of the user's body, including their eyes, and processing circuitry to analyze an input received in response to the presented images to determine one or more health conditions or defects. However, this disclosure measures intraocular pressure using backscattered light that is coming from one portion of the eye, and it uses classical methods to create an indent in the eye with an air puff and then measures the intraocular pressure using OCT imaging system. Also, since a controlled vacuum/pressure environment is not used in the disclosure during the measurement, the resulting readings may not be optimal.

Another related reference WO2018174835 discloses a glaucoma treatment eyeglass with visual stimulation consisting of the parts of visually stimulating screen, pressure control unit, electronic control and communication unit, information display, pressure control valve, eyeglass cavity pressure sensor, flexible sealing gasket, pressure control buttons, electronic control buttons, pressure pipes, external pressure control unit, actively stimulating light source, mini notice screen, mobile device and notice lamp, which patients with glaucoma eye disease may use during the day, increasing the ocular flow of blood by means of visual stimulus according to the condition and level of the disease, decreasing the neurodegenerative effects and ensuring pressure control. However, the eyeglass is a treatment device and does not have an IOP measurement function. The pressure control system around the eye cavity is used only for therapeutic purposes.

Related reference US 2015164321 discloses an eye-mountable device for measuring an intraocular pressure. The device includes a transparent polymeric material having a concave surface configured to be removably mounted over a corneal surface of an eye, an antenna, an expandable member, a sensor, and control electronics at least partially embedded in the transparent polymeric material. The expandable device is configured to expand and apply a force to the corneal surface and the sensor is configured to detect a resistance to deformation of the cornea in response to the applied force. The resistance to deformation of the cornea in response to the force applied by the expandable member is indicative of the intraocular pressure of the eye. However, this disclosure measures IOP with a contact lens, which requires contact to the eye.

Related reference WO 2017035406 discloses an apparatus for at least one of diagnosing or treating an eye condition includes a goggle enclosure, sized and shaped to be seated on an eye socket of an eye to provide one or more cavities within the enclosure that extend about an entire exposed anterior portion of the eye, a pump, in fluidic communication with the one or more cavities to apply a fluid pressure to the one or more cavities, the pump configured to adjust a fluid pressure within the one or more cavities of the goggle enclosure, and a control circuit, including a data interface to receive data directly or indirectly indicating at least one of an intraorbital pressure, ICP, IOP, or a relationship between ICP and IOP, and based on processing the received data as a feedback control variable, controlling the pump to adjust the fluid pressure within the one or more cavities, the controlling including using further monitoring of the received data to control the pump. This disclosure measures the blood flow in order to find the correct pressure value for the treatment, measures the axonal transport in order to find the correct/optimum pressure value for the treatment, and creates optimum translaminar pressure difference. However, it still requires fluid contact of the eye, which may be uncomfortable to the user's eyes and may cause infection.

Related reference WO 2019175679 discloses an ophthalmological analysis system including an air-puff generating device configured to apply an air-puff to a user's at least one eye, and at least one sensors board configured to detect and record deformation of a cornea of the user during the air-puff; wherein the detection is a three dimensional (3D) active stereo detection, the sensors board further configured to find two applanation points and hysteresis of the cornea and to calculate intraocular pressure in the user's at least one eye accordingly. However, the air-puff and the classical applanation method will require direct contact to the eye and cause uncomfortableness to the users.

Another reference WO 2021053452 discloses a system for determining an IOP of a subject includes a pressurizing device for applying pressure of varying magnitude over an external surface of an eye of the subject; a monitoring device for monitoring internal vasculature of the eye and vasculature on or around the eye; and a processing unit for correlating a first pressure or pressure range with pulsation or collapse of the internal vasculature of the eye and a second pressure or pressure range with pulsation or collapse of the vasculature on or around the eye to thereby derive the IOP of the subject. However, this invention measures the intraocular pressure by measuring the pulsation of the vessels on the eye, which heavily relies on the assumption that the pulsation of the vessels on the eye changes with the intraocular pressure.

As a result, developing non-contact, wearable, and easy-to-use intraocular measurement devices are crucial for the early diagnosis and following the disease progression.

### SUMMARY

In this respect, before explaining at least one embodiment in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

One purpose of the instant invention is to provide a solution for the above deficiency and improve the users experience in conventional tonometer. The approach is to adjust air pressure around the eyes and indirectly measure the deflection of the eyes for determining the intraocular pressure. A non-invasive technique with a wearable device is described for measuring the intraocular pressure (IOP). This solves the aforementioned problems in the conventional intraocular pressure measurement such as direct contact with the eye, difficult to use, and requiring long training to get correct measurements.

In one embodiment, the instant invention discloses a system for measuring intraocular pressure including a wearable device configured to be worn by a user and forms an air chamber between the wearable device and both eyes of the user. The wearable device includes flexible material around the edges to seal the air chamber around eye cavities of the user, and the wearable device further includes imaging sensors for collecting 2D and 3D data of the user's eyes. The wearable device is further connected with a pressure controller for controlling an air pump to modify an air pressure inside the air chamber. In one embodiment, the pressure controller is embedded on the wearable device, and in another embodiment, the pressure controller is connected to the wearable device through a flexible pressure pipe.

The system for measuring intraocular pressure further includes a central controller comprising a processor and a memory, the memory stores a computer readable instruction, and when the computer readable instruction is executed by the processor, this causes the central controller to construct a model of the user's eyes using 3D imaging techniques based on the collected 2D and 3D data and outputs an intraocular pressure reading based on the model.

In one embodiment, the system includes a lightweight monitoring device configured to collect 2D and 3D data from the user's eyes without the air chamber. The monitoring device is further configured to wirelessly communicate with the wearable device and obtain the 2D and 3D data collected from the wearable device as reference data. In one implementation, the monitoring device is a dedicated wearable hardware such as an eyeglass equipped with sensors. Alternatively, the monitoring device is a smart electronic device (i.e. smartphone, tablet, or portable camera) embedded with 3D imaging technology.

A user can use the wearable device once every period (i.e. every day, every week, or every month, etc.) to obtain an IOP measurement periodically. Between each periodically measurements of using the wearable device, the user can use the monitoring device to collect 3D images and data of the eye globe and cornea, and continue estimating IOP readings by comparing the IOP to reference data taken by the wearable device. The reference data can be stored on a cloud-based server or on a memory of the monitoring device.

In one embodiment, the imaging sensors include at least one of: stereo imaging sensors, light detecting sensors, temperature detecting sensors, acoustic sensors, electro optic sensors, lidar sensors, pressure sensors, a force sensor, a position sensor, ultraviolet sensors, and piezo crystals for ultrasonic topography. The positions and orientations of the imaging sensors can be fixed and/or adjustable to fit a user's eyes.

Further, the wearable device includes at least one of: a light emitting diode, an ultraviolet light source, a laser scanning device, a display screen for virtual reality, a display screen for augmented reality, and a scheimpflug camera.

In one aspect, the central controller further records air pressure change data by the pressure controller and associates the collected 2D and 3D data by the wearable device to the air pressure change data. Furthermore, the imaging sensors continuously collects the 2D and 3D data during which different air pressure is applied on the air chamber (i.e. the pressure controller increases and decreases the air pressure during the collecting 2D and 3D data) and the central controller continuously outputs calculated intraocular pressure values.

In one embodiment, the 2D and 3D data includes physical parameters of the user. The physical parameters include at least one of: the cornea diameter, cornea shape, color difference, color change, heat changes, viscosity of the cornea, iris shape, and iris diameter change.

In one embodiment, the central controller is further configured to determine at least one of: biomechanics of a frontal segment, a physical difference of the eye globe, conjunctiva, sclera, or cornea under varying pressure, and a reaction of the eye globe, conjunctiva, sclera, or cornea under varying pressure.

In one embodiment, the central controller is configured to separate the collected 2D and 3D data into left eye data and right eye data; and the central controller is further configured to determine the differences between the two eyes based on the left eye data and the right eye data.

In one embodiment, the wearable device further includes a display screen, and the display screen provides instructions to guide the user's eyes to a specific position.

In one embodiment, the central controller further outputs at least one of: an intracranial pressure, a translaminar pressure gradient, a central retinal artery pressure, and an ocular perfusion pressure by measuring and calculating the ocular pulsation amplitude and period under varying external pressure.

In a further embodiment of the instant invention, a method for measuring intraocular pressure is disclosed. The method comprises: forming an air chamber between a wearable device and both eyes of a user; the wearable device includes flexible material around the edges to seal the air chamber around eye cavities of the user; the wearable device further includes imaging sensors for collecting 2D and 3D data of the user's eyes; controlling, a pressure controller, an air pump to modify an air pressure inside the air chamber; wherein the pressure controller is embedded on the wearable device or is connected to the wearable device through a flexible pressure pipe; and constructing, by a central controller, a model of the user's eyes using 3D imaging techniques based on the collected 2D and 3D data and outputs an intraocular pressure calculations/estimations based on the model.

### BRIEF DESCRIPTION OF THE DRAWINGS

In the drawings, various embodiments of the present systems and methods are illustrated by way of example. It is to be expressly understood that the description and drawings are only for the purpose of illustration.

FIG. 1 illustrates a main wearable device for measuring intraocular pressure under varying external pressure.

FIG. 2 illustrates a front view of the main wearable device with a pressure control system.

FIG. 3 illustrates a side view of the main wearable device with a pressure control system.

FIG. 4 illustrates a pressure control unit embedded on the main wearable device.

FIG. 5 illustrates the head band and flexible sealing material on the main wearable device.

FIG. 6 illustrates a front, top, and side view of the main wearable device.

FIG. 7 illustrates an internal view of the main wearable device.

FIG. 8 illustrates a monitoring device without external pressure control.

FIG. 9 illustrates an internal view of the monitoring device having sensor and excitation components on the monitoring device.

FIG. 10 illustrates another alternative for the monitoring device.

FIG. 11 illustrates another alternative for daily measurements using a smart device that is already in possession of the patient.

FIG. 12 illustrates a potential use case of the disclosed intraocular pressure measurement and monitoring system.

FIG. 13 illustrates the shapes of the cornea and eye globe shape under varying pressure.

FIG. 14 illustrates an example graph of the external pressure and cornea/sclera diameter.

FIG. 15 illustrates an exemplary ocular pulsation of a user.

### DETAILED DESCRIPTION

The invention describes a new wearable tonometry device that is used to measure the intraocular pressure. This device observes the variations on the cornea and the eye globe under varying pressure conditions and measures the intraocular pressure. The working principle of the device is based on the mechanical effect of the intraocular pressure on the eye globe.

In the following description, for the purposes of explanation, numerous specific details are set forth in order to provide a thorough understanding of the embodiments of the invention. It is apparent, however, to one skilled in the art that the embodiments of the invention may be practiced without these specific details or with an equivalent arrangement.

The instant invention combines the advantages of the continuous IOP monitoring and the benefits of non-contact tonometry. The instant invention brings a new type of tonometry to the industry—pressure varying non-contact wearable tonometry. The invention is a wearable and easy-to-use device in a home environment for individual users to perform IOP measurements. Furthermore, the invention is portable and does not require the use of eye drops. Thus, the invention is particularly suitable for children and non-cooperative patients.

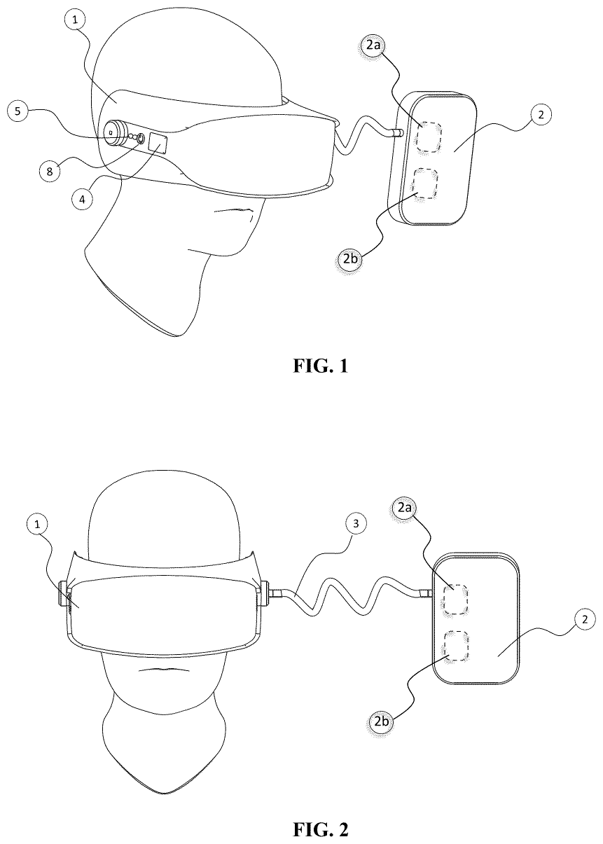

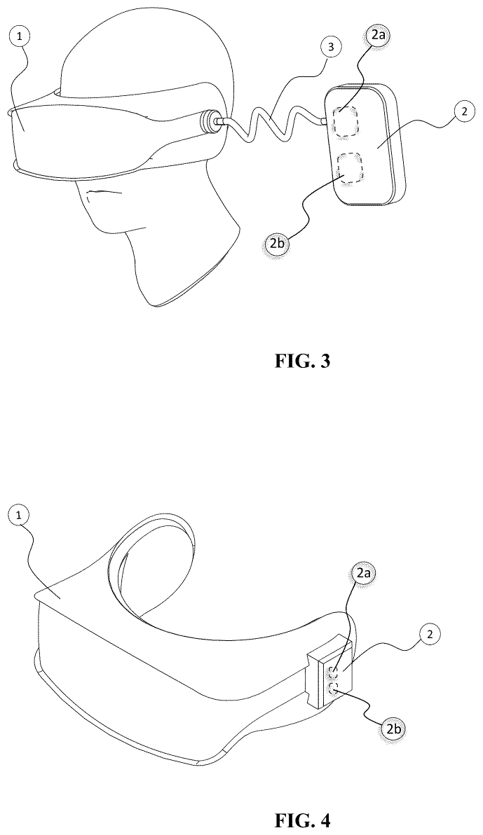

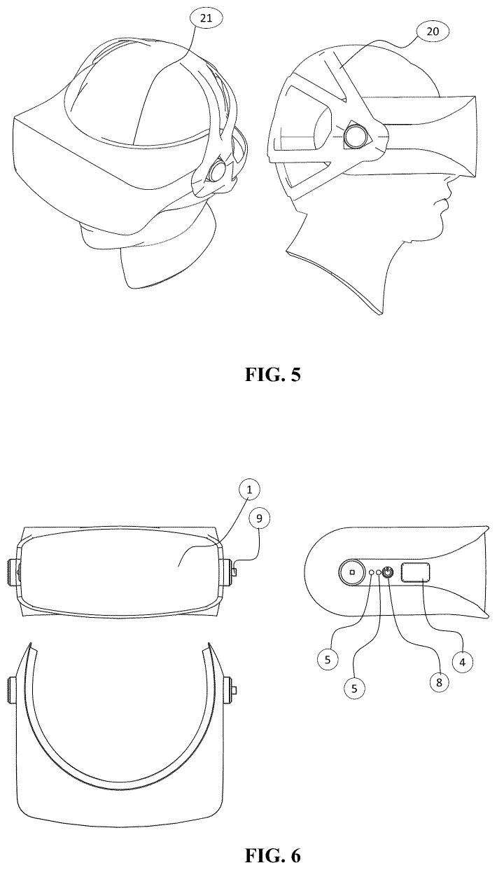

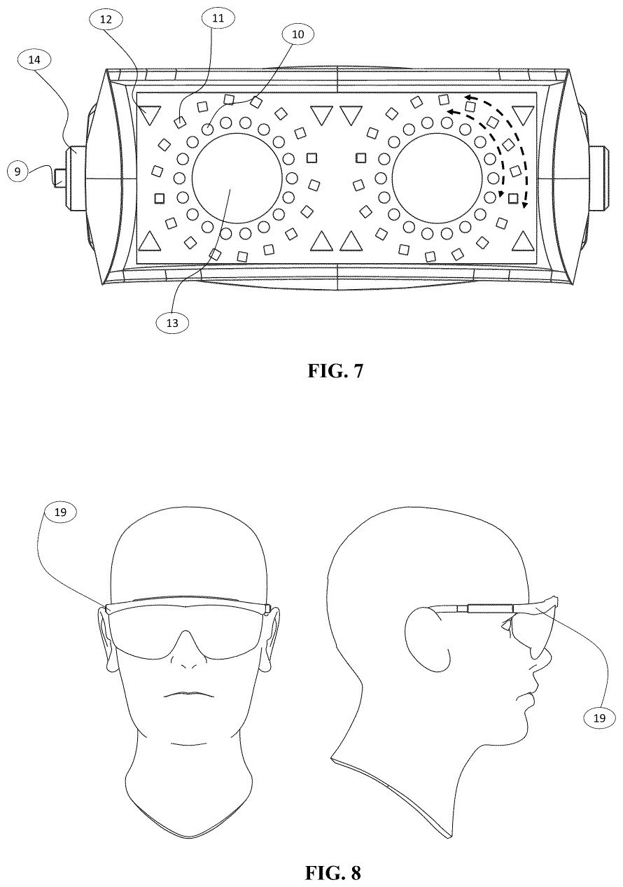

FIGS. 1-3 illustrates front and side views of a system for measuring intraocular pressure in one embodiment of the instant invention. The system includes a main wearable device 1 configured to be worn by a user and forms an air chamber between the main wearable device 1 and both eyes of the user. The main wearable device 1 includes a mini notification screen 4, notification LEDs 5, an on/off switch 8, and flexible material (shown later in FIG. 5) around the edges to seal the air chamber around eye cavities of the user. The main wearable device 1 further includes imaging sensors (shown later in FIG. 7) for collecting 2D and 3D data of the user's eyes. The 2D and 3D data includes physical parameters of the user, including, but is not limited to, the cornea diameter, cornea shape, color difference, color change, heat changes, viscosity of the cornea, iris shape, and iris diameter change. The main wearable device 1 is further connected with a pressure controller 2 for controlling an air pump 2a to modify an air pressure inside the air chamber. As shown in FIGS. 1-3, the pressure controller 2 is connected to the main wearable device 1 through a flexible pressure pipe 3. FIG. 4 illustrates another embodiment that the pressure controller 2 is embedded on the main wearable device 1. The pressure controller controls an air pump 2a to increase and decrease an air pressure inside the air chamber.

The system for measuring intraocular pressure further includes a central controller 2b in the pressure controller 2. The central controller 2b comprises a processor and a memory, the memory stores a computer readable instruction, and when the computer readable instruction is executed by the processor, causes the central controller 2b to construct a model of the user's eyes using 3D imaging techniques based on the collected 2D and 3D data and output an intraocular pressure reading based on the model. The 3D imaging techniques include, but are not limited to, Stereo imaging, Lidar, RGBD cameras, laser scanners, and time of flight sensors. In one embodiment, at least two of the above 3D imaging techniques are implemented, so that data coming from different types of sensors will be aggregated and analyzed to form high resolution 3D structure of the eyes.

The 2D and 3D data can be the image of the eye coming from a camera, or two-dimensional data coming from temperature sensors, stereo imaging sensors, light detecting sensors, temperature detecting sensors, acoustic sensors, electro optic sensors, lidar sensors, pressure sensors, a force sensor, a position sensor, ultraviolet sensors, and piezo crystals for ultrasonic topography. The 2D data defines the surfaces of the eyes for 3D reconstruction of the eyes, and the 3D data defines the shape of the eye. Under different pressure conditions, the 3D shape of the eye will be measured. The aggregated and analyzed data including and merging the data coming from various sensors will be used to calculate the intraocular pressure. Based on the 2D and 3D data, the central controller 2b is designed to calculate biomechanics of a frontal segment, a physical difference of the eye globe, conjunctiva, sclera, or cornea under varying pressure, and a reaction of the eye globe, conjunctiva, sclera, or cornea under varying pressure. The central controller 2b further separates the collected 2D and 3D data into left eye data and right eye data. The central controller 2b then calculates differences of a variety of parameters between the two eyes based on the left eye data and the right eye data. In one embodiment, the central controller 2b is embedded inside the main wearable device 1 (i.e. inside a frame of the main wearable device). In another embodiment, the central controller 2b is implemented as a server communicating wirelessly with the main wearable device 1.

FIG. 5 shows an adjustable head band 20 to fix the main wearable device 1 to a user's head and flexible sealing material 21 to seal the air chamber around eyes to enable pressure control of the instant invention. FIG. 6 illustrates a front, top, and side view of the main wearable device 1, including the mini notification screen 4, two notification LEDs 5, the on/off switch 8, and a pressure pipe outlet 9. The mini notification screen 4 allows the main wearable device 1 to show a most current intraocular pressure reading of the user. The two notification LEDs 5 are designed to indicate on, off, measuring, error states of the main wearable device 1. For example, a green light can be used to show the main wearable device 1 is powered on, no light can mean the main wearable device 1 is powered off, a blinking green light can show the main wearable device 1 is currently performing measurements, and a blinking red light can indicate the main wearable device 1 is in an error state. In another embodiment, other LED light schemes can be used to identify the various different operation states of the main wearable device 1. Furthermore, the pressure pipe outlet 9 is an air outlet connecting the air chamber to the pressure controller 2 with the flexible pressure pipe 3.

FIG. 7 illustrates the inside view of the main wearable device 1. As shown in FIG. 7, the main wearable device 1 includes the pressure pipe outlet 9 as discussed above, a stimulator array 10, a receiver sensor array 11, pressure sensors 12, a display 13, and a pressure inlet controller 14. The stimulator array 10 are sources that include, but are not limited to, LEDs, light, heat, sound, electric, scanner, infrared, and ultraviolet sources. The emissions of the stimulator array 10 is controlled by the central controller 2b. The receiver sensor array 11 are sensors for detecting a various aspect of the user's eyes, including, but are not limited to scheimpflug cameras, stereo imaging sensors, light detecting sensors, temperature detecting sensors, acoustic sensors, electro optic sensors, lidar sensors, pressure sensors, a force sensor, a position sensor, ultraviolet sensors, and piezo crystals for ultrasonic topography. The data collected by the receiver sensor array 11 is transmitted to the central controller 2b for further processing. The pressure sensors 12 are functioned to sense the air pressure inside the air chamber. Readings by the pressure sensors 12 are transmitted to the pressure controller 2 for reflecting a current air pressure inside the air chamber. All components can be fixed and/or change their positions and orientations in the device to take different measurements from different positions or different angles. The position and orientation of each element of the stimulator array 10, the receiver sensor array 11, and the pressure sensors 12 are adjustable by the user to take different measurements from different positions or different angles. The display 13 of the main wearable device 1 allows showing of virtual reality (VR) content, augmented reality (AR) content, or instructions to guide the user's eyes for focusing on specific points, thereby increasing the accuracy of the measurements. Moreover, the pressure inlet controller 14 enables the user to manually open, close, and adjust the pressure inlet.

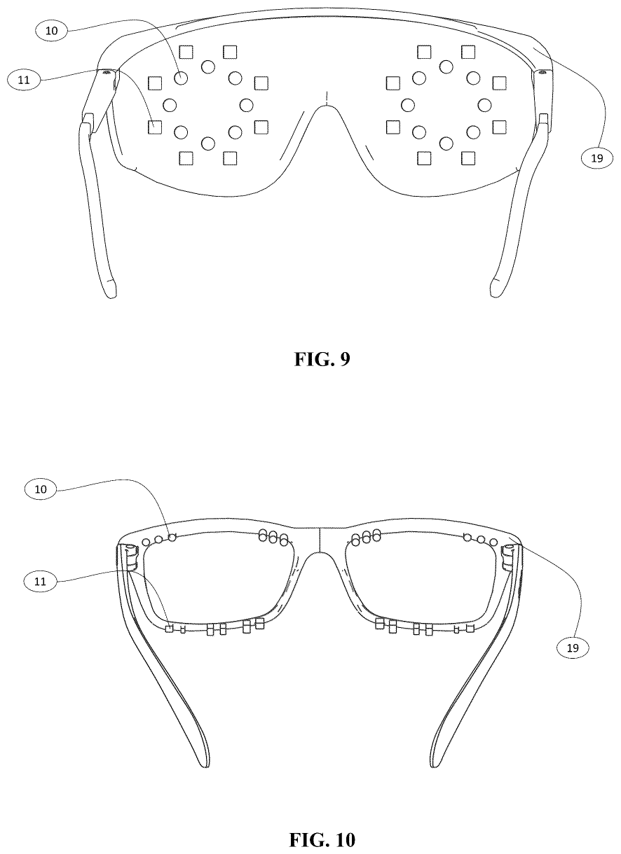

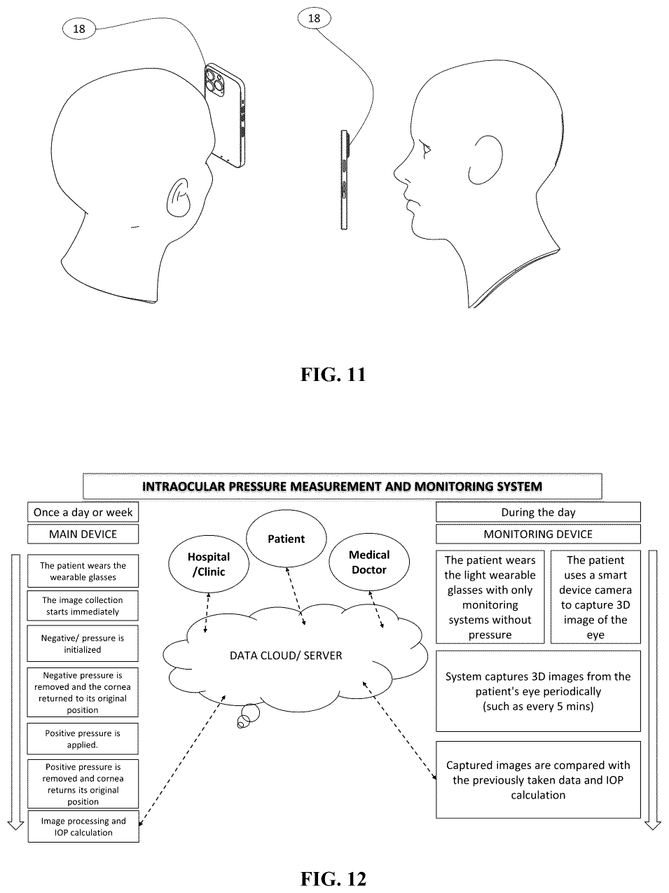

FIG. 8 illustrates another embodiment of the instant invention. A lightweight monitoring device 19 is configured to collect 2D and 3D data from the user's eyes without the air chamber. As shown in FIG. 8, the monitoring device 19 has outer appearance as a dedicated eyeglass. The monitoring device is further configured to wirelessly communicate with the main wearable device and obtain the 2D and 3D data collected from the main wearable device as reference data. FIG. 9 illustrates the inside of the monitoring device 19, which includes a stimulator array 10 and a receiver sensor array 11 for collecting 2D and 3D data from the user's eyes. Thus, a user can use the main wearable device 1 once every period (i.e. every day, every week, or every month, etc.) to obtain an IOP measurement periodically. Between each periodic measurement using the main wearable device 1, the user can use the monitoring device 19 to collect 3D images and data of the eye globe and cornea, and continue estimating IOP readings by comparing the IOP to the reference data previously taken by the main wearable device 1. FIG. 10 shows an alternative design of the monitoring device 19. A stimulator array 10 and a receiver sensor array 11 are positioned on the frame of the eyeglass, so that the arrays 10 and 11 would not block the user's sight, thereby enabling long-term wearing the monitoring device 19 for automatically and continuously monitoring of the intraocular pressure of the user's eyes. FIG. 11 illustrates another alternative for the monitoring device 19. Instead of using monitoring devices in the shape eyeglasses, users can use their smart device sensors 18 to collect data (i.e. using the camera on the user's phone to take images) from their eye globes and transmit the collected data to the central controller 2b for calculation and estimation of the intraocular pressure based on a previously recorded reference data via the main wearable device 1.

FIG. 12 illustrates a potential use case of the intraocular pressure measurement and monitoring system of the instant invention. A patient, a medical doctor, and a hospital/clinic are connected to a cloud server in a network. The patient periodically (once a day or once a week) takes a measurement of the IOP via the main wearable device 1 (i.e. main device). The measurement process via the main wearable device includes (1) the patient wears the main wearable device, (2) the image collection starts, (3) negative pressure is initialized, (4) negative pressure is removed and the user's cornea is returned to its original position, (5) positive pressure is initialized, (6) positive pressure is removed and the user's cornea is returned to its original position, (7) the image collection ends and the collected image data is processed to obtain an IOP reading. The collected image data and IOP reading are indicated as reference data and are further transmitted to the cloud server accessible to the patient, the medical doctor, and the hospital/clinic. Once the patient has reference data store on the cloud server, the patient can choose to take another measurement of IOP using the monitoring device as shown in FIGS. 8-11. The measurement process via the monitoring device includes (1) the patient wears the light wearable glasses of FIGS. 8-10 or uses a smart device camera to capture 2D and 3D image of the eye as shown in FIG. 11, (2) if the wearable glasses of FIGS. 8-10 are used, the 3D images are captured from the patient's eyes periodically, such as every 5 minutes, and (3) the captured images are compared with the previously taken reference data to calculate and generate an current IOP reading. The IOP reading(s) is/are marked by the date and time of taking the measurement and is/are transmitted to the cloud server accessible to the patient, the medical doctor, and the hospital/clinic.

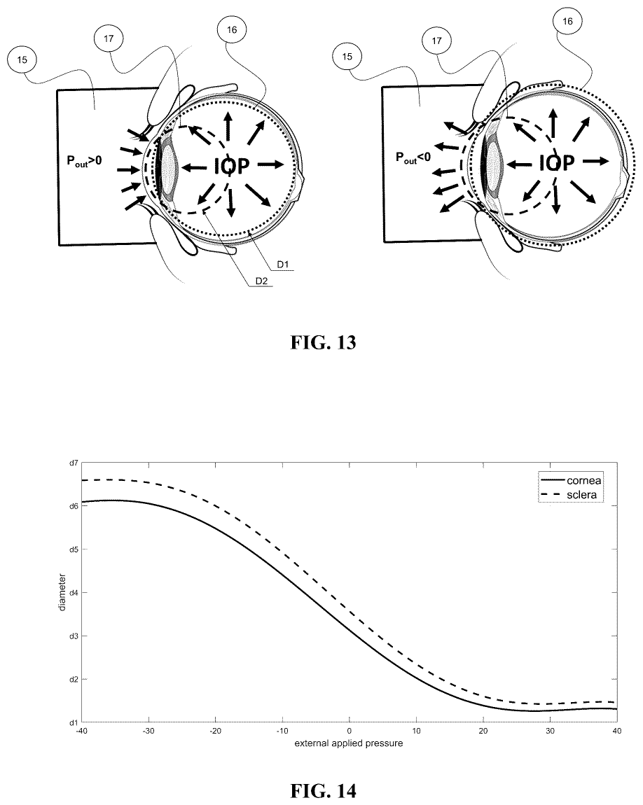

FIG. 13 illustrates the shapes of the cornea and eye globe shape under varying pressure, including air chamber 15, new eye globe shape under varying pressure 16, and new cornea shape under varying pressure 17. The left figure illustrates when positive pressure is applied in the air chamber 15, and the right figure illustrates when negative pressure is applied in the air chamber 15. D1 and D2 are the diameters of the eye globe and the cornea, respectively. Under varying external pressure, the shapes of the eye globe and cornea can be measured with 3D data collection and the diameters can be calculated. The differences between different variables are analyzed to estimate the required parameters including, but are not limited to, an intraocular pressure, an intracranial pressure, a translaminar pressure gradient, a central retinal artery pressure, and an ocular perfusion pressure by measuring and calculating the ocular pulsation amplitude and period under varying external pressure.

FIG. 14 illustrates an example graph of the external pressure and cornea/sclera diameter. To establish a relationship between the external pressure and cornea/sclera diameter, the central controller 2b further records air pressure change data by the pressure controller 2 and associates the collected 2D and 3D data by the main wearable device 1 to the air pressure change data. Once a relationship between the external pressure and cornea/sclera diameter is established for a user, the user's IOP can be estimated using this relationship.

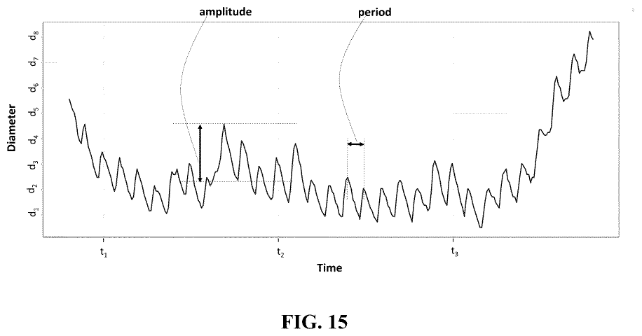

FIG. 15 illustrates an exemplary ocular pulsation of a user. The ocular pulsation amplitude and period is further calculated based on the ocular pulsation of the user derived from the collected 2D and 3D image data and data coming from the other sensors such as pressure sensors and temperature sensors.

The monitoring device 19 as shown in FIGS. 8-10 may be, for example, a smartphone, tablet, PDA, or smartwatch having a processor, computer readable media that stores software and application programs for execution by the processor, a camera for capturing images, and a wireless communication interface for communication to a network.

The term “computer-readable media” or “computer-readable medium” as used herein refers to any media/medium that participates in providing instructions to the processor for execution. Such a medium may take many forms, including but not limited to non-volatile media, volatile media, and transmission media. Non-volatile media may include, for example, optical or magnetic disks. Volatile media may include a dynamic memory. Transmission media may include coaxial cables, copper wire and fiber optics. Transmission media may also take the form of acoustic, optical, or electromagnetic waves, such as those generated during Radio Frequency (RF) and infrared (IR) data communications. Common forms of computer-readable media may include, for example, a floppy disk, a flexible disk, hard disk, magnetic tape, any other magnetic medium, a CD-ROM, a Compact Disc-Rewritable (CDRW), a Digital Video Disk (DVD), any other optical medium, punch cards, paper tape, optical mark sheets, any other physical medium with patterns of holes or other optically recognizable indicia, a RAM, a PROM, and EPROM, a FLASH-EPROM, any other memory chip or cartridge, a carrier wave, or any other medium from which a computer can read.

Those skilled in the art will appreciate that the herein described systems and methods may be subject to various modifications and alternative constructions. There is no intention to limit the scope of the invention to the specific constructions described herein. Rather, the herein described systems and methods are intended to cover all modifications, alternative constructions, and equivalents falling within the scope and spirit of the invention and its equivalents.

What is claimed is:

1. A system for measuring intraocular pressure, comprises:
a wearable device configured to be worn by a user and forms an air chamber between the wearable device and both eyes of the user; the wearable device includes flexible material around edges to seal the air chamber around eye cavities of the user; the wearable device further includes imaging sensors for collecting 2D and 3D data of the user's eyes;
a pressure controller configured to control an air pump to modify an air pressure inside the air chamber; wherein the pressure controller is embedded on the wearable device or is connected to the wearable device through a flexible pressure pipe; and
a central controller including a processor and a memory, the memory stores a computer readable instruction, and when the computer readable instruction is executed by the processor, causes the central controller to construct a model of the user's eyes using 3D imaging techniques based on the collected 2D and 3D data and outputs an intraocular pressure reading based on the model.

2. The system according to claim 1, wherein the imaging sensors include at least one of: stereo imaging sensors, light detecting sensors, temperature detecting sensors, acoustic sensors, electro optic sensors, lidar sensors, pressure sensors, a force sensor, a position sensor, ultraviolet sensors, and piezo crystals for ultrasonic topography.

3. The system according to claim 1, wherein the wearable device further includes at least one of: a light emitting diode, an ultraviolet light source, a laser scanning device, a display screen for virtual reality, a display screen for augmented reality, and a scheimpflug camera.

4. The system according to claim 1, wherein the central controller further records varying air pressure data by the pressure controller and associates the collected 2D and 3D data by the wearable device to the varying air pressure data.

5. The system according to claim 1, wherein the imaging sensors continuously collects the 2D and 3D data and the central controller continuously outputs intraocular pressure readings.

6. The system according to claim 1, wherein positions and orientations of the imaging sensors are fixed or adjustable sensors.

7. The system according to claim 1, wherein the pressure controller increases and decreases the air pressure during the collecting 2D and 3D data and during collecting data coming from all embedded sensors.

8. The system according to claim 1, wherein the 2D and 3D data includes physical parameters of the user, wherein the physical parameters include at least one of: a cornea diameter, a cornea shape, a color difference, a color change, heat changes, a viscosity of a cornea, an iris shape, and an iris diameter change.

9. The system according to claim 1, wherein the central controller is further configured to determine at least one of: biomechanics of a frontal segment, a physical difference of an eye globe, a conjunctiva, sclera, or cornea under varying pressure, and a reaction of the eye globe, conjunctiva, sclera, or cornea under varying pressure.

10. The system according to claim 1, wherein the central controller is configured to separate the collected 2D and 3D data into left eye data and right eye data; and the central controller is further configured to determine the differences between the two eyes based on the left eye data and the right eye data.

11. The system according to claim 1, wherein the wearable device further includes a display screen, and the display screen provides instructions comprising a guide for guiding the user's eyes to a specific position.

12. The system according to claim 1, wherein the central controller further outputs at least one of: an intracranial pressure, a translaminar pressure gradient, a central retinal artery pressure, and an ocular perfusion pressure by measuring and calculating an ocular pulsation amplitude and period under varying external pressure.

13. The system according to claim 1, wherein the 2D and 3D data are stored in the pressure control unit or the wearable device, and the 2D and 3D data is used to calculate and estimate the intraocular pressure.

14. A system for measuring intraocular pressure, comprises:
a monitoring device configured to collect 2D and 3D data from a user's eyes without an air chamber; the monitoring device is further configured to wirelessly communicate with a wearable device and obtain 2D and 3D data collected from the wearable device as reference data; wherein the monitoring device is a dedicated wearable hardware or a smart electronic device embedded with 3D imaging technology.

15. A method for measuring intraocular pressure, comprises:
forming an air chamber between a wearable device and both eyes of a user; the wearable device includes flexible material around edges to seal the air chamber around eye cavities of the user; the wearable device further includes imaging sensors for collecting 2D and 3D data of the user's eyes;
controlling, by a pressure controller, an air pump to modify an air pressure inside the air chamber; wherein the pressure controller is embedded on the wearable device or is connected to the wearable device through a flexible pressure pipe; and
constructing, by a central controller, a model of the user's eyes using 3D imaging techniques based on the collected 2D and 3D data and outputting an intraocular pressure reading based on the model.

16. The method according to claim 15, wherein the imaging sensors include at least one of: stereo imaging sensors, light detecting sensors, temperature detecting sensors, acoustic sensors, electro optic sensors, lidar sensors, pressure sensors, a force sensor, a position sensor, ultraviolet sensors, and piezo crystals for ultrasonic topography.

17. The method according to claim 15, wherein the wearable device further includes at least one of: a light emitting diode, an ultraviolet light source, a laser scanning device, a display screen for virtual reality, a display screen for augmented reality, and a scheimpflug camera.

18. The method according to claim 15, wherein the 2D and 3D data includes physical parameters of the user, wherein the physical parameters include at least one of: a cornea diameter, a cornea shape, a color difference, a color change, heat changes, a viscosity of a cornea, an iris shape, and an iris diameter change.

19. The method according to claim 15, wherein the central controller is further configured to determine at least one of: biomechanics of a frontal segment, a physical difference of an eye globe, a conjunctiva, sclera, or cornea under varying pressure, and a reaction of the eye globe, conjunctiva, sclera, or cornea under varying pressure.

20. A non-transitory computer readable medium storing computer readable instructions, wherein when the computer readable instructions are executed by at least one processor, cause the at least one processor to perform the method according to claim 15.
